(19) United States
(12) Patent Application Publication
(10) Pub. No.: US 2018/0361002 A1
(43) Pub. Date: Dec. 20, 2018

(54) Sanitizing Assembly

(71) Applicant: Ashlea Mastrocola, King Of Prussia, PA (US)

(72) Inventor: Ashlea Mastrocola, King Of Prussia, PA (US)

(21) Appl. No.: 15/622,908

(22) Filed: Jun. 14, 2017

(51) Int. Cl. A61L 2/18; B05B 9/00; B05B 9/04; B64D 11/00

(52) U.S. Cl. CPC A61L 2/18; B05B 9/007; A61L 2202/15; B64D 11/00; B05B 9/0423

(57) ABSTRACT

A sanitizing assembly for sanitizing passenger seats of an airplane includes a power module, a pump, a tank, and plurality of dispensers. The power module, the pump, the tank, and the plurality of dispensers are coupled to and positioned in an airplane. The pump is operationally coupled to the power module. The tank is fluidically coupled to the pump. The tank is configured to store a sanitizer solution. The plurality of dispensers is fluidically coupled to the pump. Each dispenser is positioned proximate to a respective seat of the airplane. The pump is positioned to compel the sanitizer solution from the tank to the plurality of dispensers. The sanitizer solution is sprayed onto the seats to sanitize the seats.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to sanitizing assemblies and more particularly pertains to a new sanitizing assembly for sanitizing passenger seats of an airplane.

### BRIEF SUMMARY OF THE INVENTION

An embodiment of the disclosure meets the needs presented above by generally comprising a power module, a pump, a tank, and plurality of dispensers. The power module, the pump, the tank, and the plurality of dispensers are coupled to and positioned in an airplane. The pump is operationally coupled to the power module. The tank is fluidically coupled to the pump. The tank is configured to store a sanitizer solution. The plurality of dispensers is fluidically coupled to the pump. Each dispenser is positioned proximate to a respective seat of the airplane. The pump is positioned to compel the sanitizer solution from the tank to the plurality of dispensers. The sanitizer solution is sprayed onto the seats to sanitize the seats.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top view of a sanitizing assembly according to an embodiment of the disclosure.

FIG. 2 is a side view of an embodiment of the disclosure.

FIG. 3 is a detail view of an embodiment of the disclosure.

FIG. 4 is a block diagram of an embodiment of the disclosure.

### DETAILED DESCRIPTION OF THE INVENTION

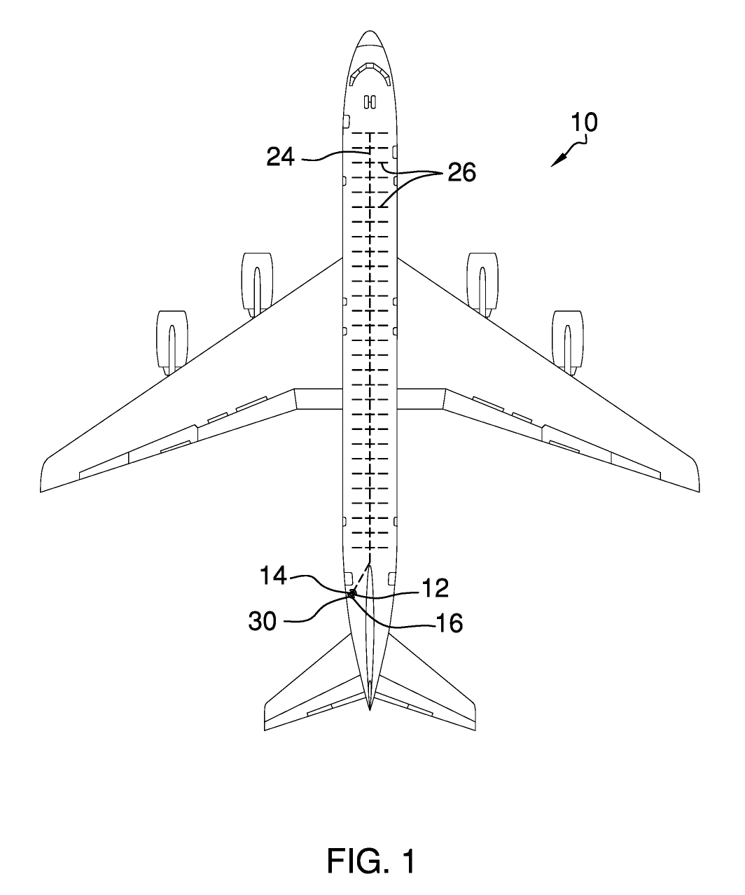

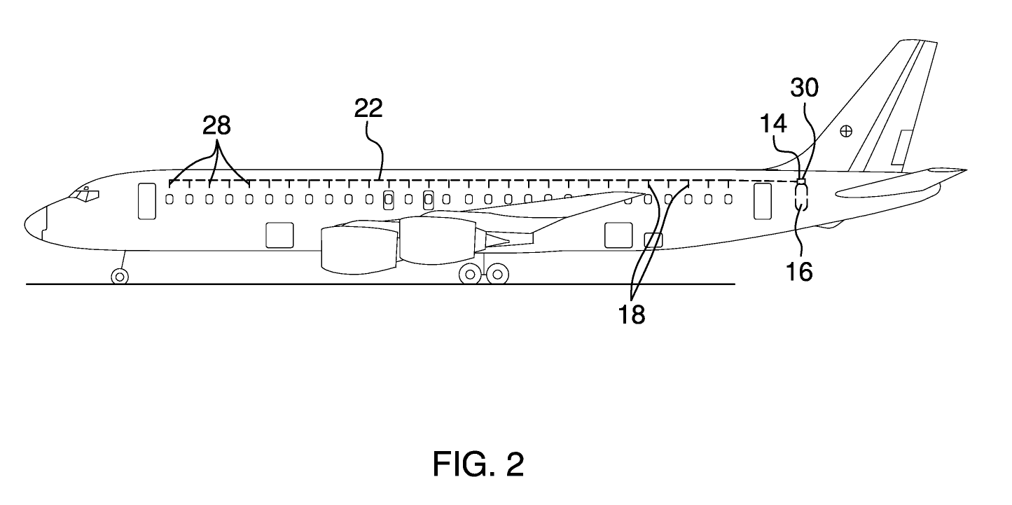

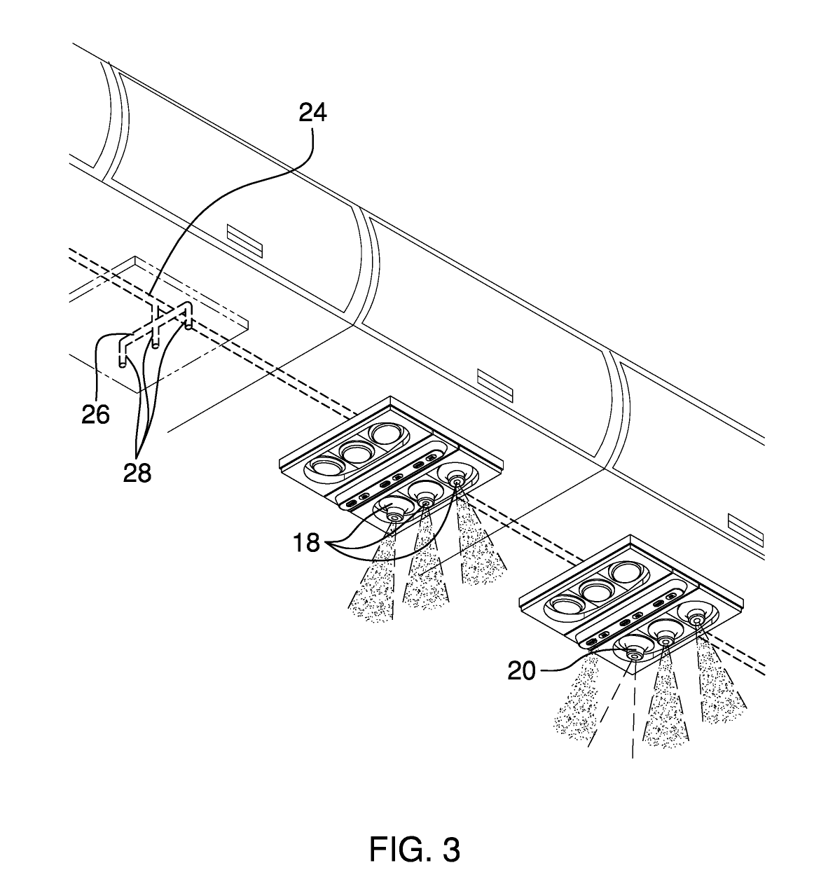

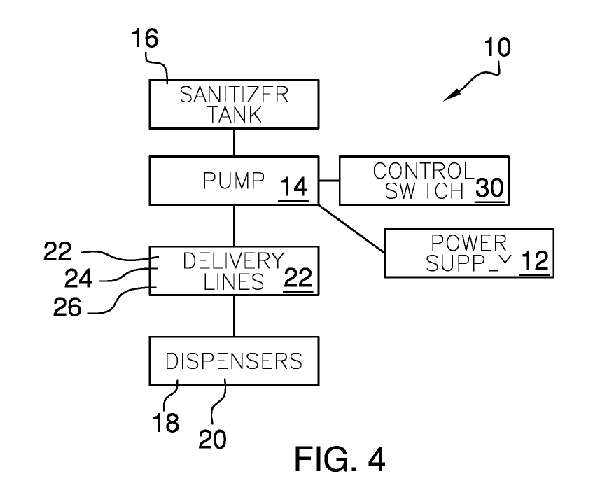

With reference now to the drawings, and in particular to FIGS. 1 through 4 thereof, a new sanitizing assembly embodying the principles and concepts of an embodiment of the disclosure and generally designated by the reference numeral 10 will be described.

As best illustrated in FIGS. 1 through 4, the sanitizing assembly 10 generally comprises a power module 12 that is coupled to and positioned in an airplane. In one embodiment, the power module 12 comprises an electrical circuit of the airplane. A pump 14 is coupled to and positioned in the airplane. The pump 14 is operationally coupled to the power module 12. A tank 16 is coupled to and positioned in the airplane. The tank 16 is fluidically coupled to the pump 14. The tank 16 is configured to store a sanitizer solution.

A plurality of dispensers 18 is coupled to and positioned in the airplane. The plurality of dispensers 18 is fluidically coupled to the pump 14. Each dispenser 18 is positioned proximate to a respective seat of the airplane. The pump 14 is positioned to compel the sanitizer solution from the tank 16 to the plurality of dispensers 18. The sanitizer solution is sprayed onto the seats to sanitize the seats. In one embodiment, each dispenser 18 comprises a gasper 20 of the respective seat.

At least one tube 22 is coupled to and extends between the pump 14 and the plurality of dispensers 18. The pump 14 is configured to compel the sanitizer solution from the tank 16 through the at least one tube 22 to the plurality of dispensers 18.

In one embodiment, the at least one tube 22 comprises at least one first pipe 24, a plurality of second pipes 26, and a plurality of third pipes 28. The at least one first pipe 24 is coupled to the pump 14 and extends longitudinally along a fuselage of the airplane. Each at least one first pipe 24 is positioned over a respective zone of seats, such as a center section in a jumbo airplane. Each second pipe 26 is coupled to and extends substantially perpendicularly from a respective at least one first pipe 24. Each third pipe 28 is coupled to and extends between a respective second pipe 26 and an associated dispenser 18.

A controller 30 is coupled to and positioned in the airplane. The controller 30 is operationally coupled to the pump 14 and the power module 12. The controller 30 is positioned to selectively couple the pump 14 to the power module 12. The pump 14 is configured to compel the sanitizer solution from the tank 16 to the plurality of dispensers 18. The sanitizer solution is sprayed onto the seats to sanitize the seats.

In use, the controller 30 is positioned to selectively couple the pump 14 to the power module 12. The at least one tube 22 is positioned on the pump 14 and the plurality of dispensers 18 so that the pump 14 is configured to compel the sanitizer solution from the tank 16 through the at least one tube 22 to the plurality of dispensers 18. The sanitizer solution is sprayed onto the seats to sanitize the seats.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A sanitizing assembly comprising:
a power module coupled to and positioned in an airplane;
a pump coupled to and positioned in the airplane, said pump being operationally coupled to said power module;
a tank coupled to and positioned in the airplane, said tank being fluidically coupled to said pump, said tank being configured for storing a sanitizer solution;
a plurality of dispensers coupled to and positioned in the airplane, said plurality of dispensers being fluidically coupled to said pump, each said dispenser being positioned proximate to a respective seat of the airplane; and
wherein said pump is positioned for compelling the sanitizer solution from said tank to said plurality of dispensers such that the sanitizer solution is sprayed onto the seats for sanitizing the seats.

2. The assembly of claim 1, further including said power module comprising an electrical circuit of the airplane.

3. The assembly of claim 1, further including each said dispenser comprising a gasper of the respective seat.

4. The assembly of claim 1, further including at least one tube coupled to and extending between said pump and said plurality of dispensers, wherein said at least one tube is positioned on said pump and said plurality of dispensers such that said pump is configured for compelling the sanitizer solution from said tank through said at least one tube to said plurality of dispensers.

5. The assembly of claim 1, further including said at least one tube comprising:
at least one first pipe, said at least one first pipe being coupled to said pump and extending longitudinally along a fuselage of the airplane such that each said at least one first pipe is positioned over a respective zone of seats, such as a center section in a jumbo airplane;
a plurality of second pipes, each said second pipe being coupled to and extending substantially perpendicularly from a respective said at least one first pipe; and
a plurality of third pipes, each said third pipe being coupled to and extending between a respective said second pipe and an associated said dispenser.

6. The assembly of claim 1, further including a controller coupled to and positioned in the airplane, said controller being operationally coupled to said pump and said power module, wherein said controller is positioned for selectively coupling said pump to said power module such that said pump is configured for compelling the sanitizer solution from said tank to said plurality of dispensers such that the sanitizer solution is sprayed onto the seats for sanitizing the seats.

7. A sanitizing assembly comprising:
a power module coupled to and positioned in an airplane, said power module comprising an electrical circuit of the airplane;
a pump coupled to and positioned in the airplane, said pump being operationally coupled to said power module;
a tank coupled to and positioned in the airplane, said tank being fluidically coupled to said pump, said tank being configured for storing a sanitizer solution;
a plurality of dispensers coupled to and positioned in the airplane, said plurality of dispensers being fluidically coupled to said pump, each said dispenser being positioned proximate to a respective seat of the airplane, wherein said pump is positioned for compelling the sanitizer solution from said tank to said plurality of dispensers such that the sanitizer solution is sprayed onto the seats for sanitizing the seats, each said dispenser comprising a gasper of the respective seat;
at least one tube coupled to and extending between said pump and said plurality of dispensers, wherein said at least one tube is positioned on said pump and said plurality of dispensers such that said pump is configured for compelling the sanitizer solution from said tank through said at least one tube to said plurality of dispensers, said at least one tube comprising at least one first pipe, a plurality of second pipes, and a plurality of third pipes, said at least one first pipe being coupled to said pump and extending longitudinally along a fuselage of the airplane such that each said at least one first pipe is positioned over a respective zone of seats, such as a center section in a jumbo airplane, each said second pipe being coupled to and extending substantially perpendicularly from a respective said at least one first pipe, each said third pipe being coupled to and extending between a respective said second pipe and an associated said dispenser;
a controller coupled to and positioned in the airplane, said controller being operationally coupled to said pump and said power module, wherein said controller is positioned for selectively coupling said pump to said power module such that said pump is configured for compelling the sanitizer solution from said tank to said plurality of dispensers such that the sanitizer solution is sprayed onto the seats for sanitizing the seats; and
wherein said controller is positioned for selectively coupling said pump to said power module, wherein said at least one tube is positioned on said pump and said plurality of dispensers such that said pump is configured for compelling the sanitizer solution from said tank through said at least one tube to said plurality of dispensers such that the sanitizer solution is sprayed onto the seats for sanitizing the seats.
